(19) United States
(12) Patent Application Publication
(10) Pub. No.: US 2003/0229947 A1
(43) Pub. Date: Dec. 18, 2003

(54) Vibrant and durable hair colouring compositions comprising non oxidative azo thiosulphate dye molecules

(76) Inventors: Colin John Clarke, Middlesex (GB); Guido Joseph Danhieux, Charlotte, NC (US); James Charles Dunbar, Morrow, OH (US); James Myron Hanna JR., Kannapolis, NC (US); Stevan David Jones, Cincinnati, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 10/308,569

(22) Filed: Dec. 3, 2002

Related U.S. Application Data
(60) Provisional application No. 60/387,549, filed on Jun. 10, 2002.

(51) Int. Cl. A61K 7/13

(52) U.S. Cl. 8/405; 8/406; 8/411

(57) ABSTRACT

The present invention relates to consumer improved hair colouring compositions, which provide improved vibrancy and durability of vibrancy, mulitfaceted colour and complete gray coverage both immediately after dyeing and over the wash and dry cycle. In particular the invention relates to hair colouring compositions comprising specified non oxidative azo thiosulphato dyes and their utilization in combination with oxidative coupler and developer dye systems.

### CROSS REFERENCE TO RELATED APPLICATION

This application claims the benefit of U.S. Provisional Application No. 60/387,549 (Case CM2678FPL), filed Jun. 10, 2002.

### FIELD

The present invention relates to dyes and their use in hair colouring compositions.

### BACKGROUND

The permanent alteration of the colour of human hair hereinafter referred to as hair by the application of hair dyes is well known.

In order to provide the consumer with the hair colour and shade and the intensity of color and shade desired, a very complex chemical process is utilized. The hair dyeing molecules are typically produced from the reaction of at least one oxidative colouring agent with an oxidizing agent to form a coloured molecule in situ on and in the hair of consumers. This typically takes place in an aggressive environment at ca pH 10 in the presence of an alkalizing agent and in the presence of an oxidizing agent. Moreover, this process is repeated regularly by the consumer in order to maintain the desired hair colour and shade and the intensity of color and shade and to ensure continual, even coverage of the hair including coverage of new hair growth.

The manufacturer of such products is also required to work within a large number of constraints. Since these products are being placed in direct contact with the consumers' skin, there exists the potential for accidental contact with the eye or for ingestion (for example), which can occur during the dyeing process. Therefore, the formulation must meet rigorous safety requirements and not cause any allergic reaction. In addition to meeting these requirements, the products must also be optically and olfactory pleasing to the consumer. In particular, the products need to meet certain physical parameters in order to ensure that the product can be easily applied to the hair by the consumer to provide the desired effect, without unintentional staining of the consumers' clothes, skin or other objects.

The manufacturer is also required to provide a large range of different resulting colours to the hair colouring consumer who may just wish to enhance the natural colour of the hair, cover grey or completely alter the hair colour to a different natural appearing hair colour or a ‘synthetic’ appearing hair colour. Consequently, the manufacture of such products is typically required to offer over twenty different formulations, of varying colors and shades to meet each consumer's specific needs. These formulations have to be individually formulated and are typically complex formulae containing a mixture of different dye compounds. As a result the manufacture of such products ranges can be costly and complex.

Permanent hair dyeing formulations typically comprise oxidative hair dye precursors, which can diffuse into the hair through the cuticle and into the cortex where they can then react with each other and suitable oxidising agents to form the end dye molecules. Due to the larger size of these resultant molecules they are unable to readily diffuse out of the hair during subsequent washing with water and/or detergents, hence delivering a consumer-desired permanency of colour.

However despite the fact that commercial hair dyeing products have been available for many years, the products still exhibit a number of consumer-related deficiencies.

A particularly critical performance area for the consumer and the hair dye formulations is their ability to effectively colour grey hair. Indeed whilst the amount of grey hair to be coloured varies considerably from consumer to consumer, the resultant overall appearance of the coloured hair demanded by the consumer should be identical for the naturally pigmented hair and the grey hair on head, with the added requirement that the initial coverage is maintained during the post dyeing washing and drying cycle.

Similarly, many commercially available products do not deliver the colour evenly along the whole length of the hair i.e. the consumer experiences a difference in color uptake at the root versus at the hair tip due to the differences in trying to colour new growth, non-damaged virgin hair and the tips of the hair, which are typically severely damaged after consumer abuse e.g. multiple dyeing cycles, physical brushing and/or combing and excess heat during hair drying.

Another un met consumer requirement area in current permanent hair dyes is their ability to deliver consumer-preferred vibrant colours, having superior shine, tonal, shade, intensity and hue qualities.

Another un-met requirement area in current permanent hair dyes is the consumer need for hair colors which do not appear artificial. In fact, in order to effectively mimic natural hair colour, the hair colourant should deliver the same colour but with a range of 2 to 12 lightness levels and 1 to 6 variations in color tone in adjacent hair fibres on head, to provide a natural appearing multi-faceted, multi-tonal hair colour. Attempts to address this consumer need have been described in the literature. For example WO00/76469 describes the combination of inorganic persulphate, hydrogen peroxide, oxidative dyes and surfactants to provide a one step method for simultaneously colouring and highlighting hair to provide variations in tone and hue.

Another consumer-need area is for the maintenance of the initially delivered hair color and shade and intensity of color and shade to the hair, which is not typically currently maintained during the post dyeing wash and drying cycle. During the course of the post dyeing wash and drying cycle due to size, conformation and water solubility differences, certain dye molecules are more readily able to diffuse out of the hair than others. This results in the loss of tone and color intensity in the hair with an overall consumer-unfavourable fading of the hair colour. This typically tends to be consumer-noticeable after just 1-2 weeks of wash and drying cycling. Consequently the hair has a duller appearance, which is highly undesirable to the consumer.

Alternatively, products which deliver extreme colour durability in hair may cause a consumer-unfavourable build-up of colour in the hair which will impact the consumer-targeted color outcome of subsequent colouring cycles and resulting in e.g. non-targeted color and shade on head, or a block, non-natural looking color on head, or too dark a color on head. This is highly undesirable to the hair colouring consumer who regularly colours.

Hence, it would be desirable to provide the consumer with a hair colourant which firstly delivers the target color and tone, and simultaneously delivers both a vibrant and multi faceted initial colour appearance which is maintained during the typical lifetime of a permanent colorant on head i.e. 3-8 weeks of post-dyeing wash and dry cycling, without any significant off toning and without any significant build-up of color in subsequent colourings.

The combination of oxidative and non oxidative so called direct dye hair colorants (e.g. acid dyes, basic dyes, non-ionic dyes, anionic dyes, cationic dyes, HC dyes) is known in the art and has been used in some commercial products in order to try and deliver target consumer color and shade and at least an initial vibrancy to the hair. However since the majority of these direct dyes are merely delivered to the surface and the cuticle of the hair, the products suffer from the problem of rapid fading of the colour and shade and intensity of color and shade during the post dyeing wash and dry cycling, resulting in a quick washed-out and off-toned appearance of the hair.

Another form of dye, which has been discussed in the literature in order to provide permanent colour typically to cellulose fibres but also to hair fibres, are reactive dyes.

Various types of reactive dyes are available for commercial use, typically in the field of textile dyeing and they differ widely in their reactivity. They range from very low reactivity to highly reactive dyes (such as Cibacron F dyes available from Ciba and Procion MX dyes available from BASF). Most of the reactive dyes are built on a similar structure consisting of (1) a “chromophore” (the color-bearing group), (2) a “reactive” group (usually a heterocyclic carbon-nitrogen ring system), and (3) a “leaving group” which is part of the “reactive” carbon-nitrogen group, which is generally a halogen atom (e.g. chlorine family). This “leaving group” on the dye is substituted during a reaction with the fiber with a functionality on the fibre (e.g. an ionised hydroxyl group). This is the point at which a covalent, permanent bond is formed between the dye and the fibre, thereby offering a means to permanency and the provision of the desired colour and vibrancy without off-toning and fade-out during the post dyeing wash and dry cycling.

Such dyes are described for example in EP 122600, EP 639237, U.S. Pat. No. 3,415,606, U.S. Pat. No. 5,493,010, WO95/25842, and WO 00/49092.

However the problem with such dyes is that in reaction with hair, the hair has minimal “reaction” sites for substitution of the dye “leaving group”, and typically requires a pre-reduction step e.g. with thioglycolic acid reducing agent, to create the required number of bonding sites for the dye molecules to react with. Such an additional step is undesirable to the consumer as it further lengthens and complicates the dyeing process and also results in additional damage to the hair.

Thus there is still a need to provide hair dye products, which can be prepared as simple formulations, which do not require a complex mixture of hair dye compounds, and which can be applied to hair with no additional steps in the dyeing process compared to the typical permanent hair dyeing process.

It has now been surprisingly found that non oxidative dyes according to the formula below, preferably utilised in combination with an oxidative dye developer and dye coupler system, provide formulations which deliver superior initial and durable color, tone and vibrancy and superior initial and durable multifaceted, multi-tonal properties along the complete hair shaft, completely covering the grey hairs, with these properties being substantially maintained until the next colouring cycle.

### SUMMARY

The present invention relates to a durable hair colouring composition comprising at least one non oxidative azo thiosulphate dye molecule, wherein said molecule has a water solubility of less than 1% at pH 5.5 and said durable composition has a delta L of less than 20, a delta C of less than 10 and a delta H of less than 10 after a 12 washing and dying cycle as defined herein.

In another aspect, the present invention relates to a durable and vibrant hair colouring composition comprising at least one non oxidative azo thiosulphate dye molecule, wherein said molecule has a molar extinction coefficient of greater than 20000 mol⁻dm³cm⁻¹ preferably at least 30000 mol⁻¹dm³cm⁻¹, more preferably at least 50000 mol⁻¹dm³cm⁻¹.

### DETAILED DESCRIPTION

The present invention relates to hair colouring compositions comprising durable cuticle-based non oxidative hair dye compounds, which do not require an oxidative environment to deliver color. The hair colouring compositions of the present invention provide superior color, color intensity and shade, and subsequent superior durability/wash fade and comprise at least one one mono azo group and at least one thiosulphate group (also referred to herein as a ‘bunte’ group) and are typically provided with a cosmetically acceptable vehicle to carry the dye compound onto or into the hair to provide colour to the hair.

Whilst not being bound by theory, it is believed that the non oxidative dyes of the present invention, due to their size, conformation, and functionalities, specifically the presence of the thiosulphate group on the azo benzene ring, are water insoluble once inside the hair. Due to their size and conformation, penetration of the hair is limited mainly to the cuticle. The dyes not only have an initial high affinity to hair but also precipitate within the hair cuticle post-washing and rinsing, and during the hair drying process. Together with the overall size of these molecules, the dyes are prevented from readily diffusing out of the hair and thereby providing increased durability over traditional commercially available dye molecules during the post-dye, wash and dry consumer cycling.

According to the present invention the dye molecule has the following formula and the salts thereof such as sodium, potassium and calcium salts thereof:

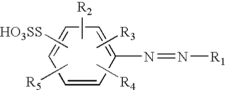

The azo thiosulphato benzene ring may be independently substituted by R2, R3, R4 and R5 groups. Each of the R2, R3, R4, and R5 groups are independently selected from:-

1. hydrogen,

2. C1-C10 linear or branched alkyl, C1-C10 linear or branched substituted alkyl, wherein the substituted groups may be selected from halogens, cyano, alkoxy, amino, amido and thiol groups,

3. C1-C10 linear or branched alkenyl and C1-C10 linear and branched alkenyl containing an oxygen or a nitrogen,

4. aryl, wherein the term aryl as used herein refers to an aromatic ring, substituted aromatic ring, coupled benzene ring systems (i.e. biaryl ring systems) substituted coupled benzene ring systems, coupled benzene ring—non aromatic ring systems, substituted coupled benzene ring—non aromatic ring systems, condensed benzoid ring systems (i.e. systems comprising 2 or more benzene rings fused together), substituted condensed benzoid ring systems, condensed benzene—non aromatic ring systems, and substituted condensed benzene—non aromatic ring systems; wherein these ring systems may have from 6 to 36 carbon atoms in the system, and the non aromatic ring components having from 3 to 8 carbon in the ring; each of substituted systems described herein above may be substituted with groups selected from for example halogens, cyano, alkyl alkoxy, hydroxy, amino, amido, thiol, nitro, nitroso, azo, alkyl sulphone, alkenyl sulphone, sulphato alkyl sulphone and sulphonate,

5. C1-C8 non aryl ring systems and substituted C1-C8 non aryl ring systems,

6. auxochromes preferably selected from NH2, NH3, COOH, HSO3 or OR1, wherein R1 is defined hereinafter, and

7. nitro, nitroso, thiosulphato, sulphonate, alkyl sulphone and alkenyl sulphone or sulphato alkyl sulphone.

R1 is selected from:

1. hydrogen,

2. C1-C10 linear or branched alkyl, C1-C10 linear or branched substituted alkyl, wherein the substitution groups are preferably hydroxy, halogens, cyano, alkoxy, amino, amido and thiol groups,

3. C1-C10 linear or branched alkenyl

4. aryl as defined hereinabove

5. C1-C8 non aryl ring systems and C1-C8 substituted non aryl ring systems

6. auxochromes preferably selected from NH2, NH3, COOH, HSO3, OH and OR wherein R is a C1-C10 linear or branched alkyl group.

Preferably R1 is according to the formula:

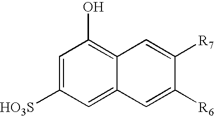

wherein R6 and R7 are independently selected from:

1. hydrogen,

2. C1-C10 linear or branched alkyl, C1-C10 linear or branched substituted alkyl,

3. C1-C10 linear or branched alkenyl,

4. Aryl as defined hereinabove

5. C1-C8 non aryl ring systems and C1-C8 substituted non aryl ring systems

6. Auxochromes preferably selected from NH2, NH3, COOH, HSO3, OH.

More preferably R6 and R7 are independently selected from hydrogen, phenylamine or a substituted phenylamine. The substituted phenylamine preferably has substituents selected from alkoxy, sulphonate, thiosulphonate NH2, NH3 and COOH. The alkoxy substituent preferably comprises a methoxy or ethoxy substituent.

A particularly preferred red dye molecule for use herein has the formula given below:

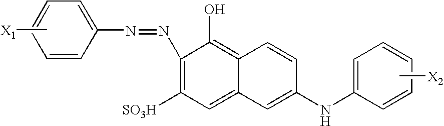

wherein X₁ is SSO₃H or a salt thereof and X₂ is H or SSO₃H or a salt thereof, most preferably H. Also useful herein is the sulphonic acid analogue thereof.

A particularly preferred brown molecule for use herein has the formula given below:

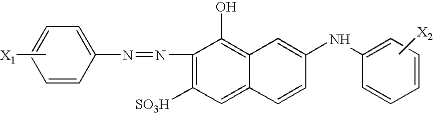

wherein X₁ is SSO₃H or a salt thereof and X₂ is H or SSO₃H or a salt thereof. Also useful herein is the sulphonic acid analogue thereof.

Other preferred molecules for use in the present invention have the formulae indicated below.

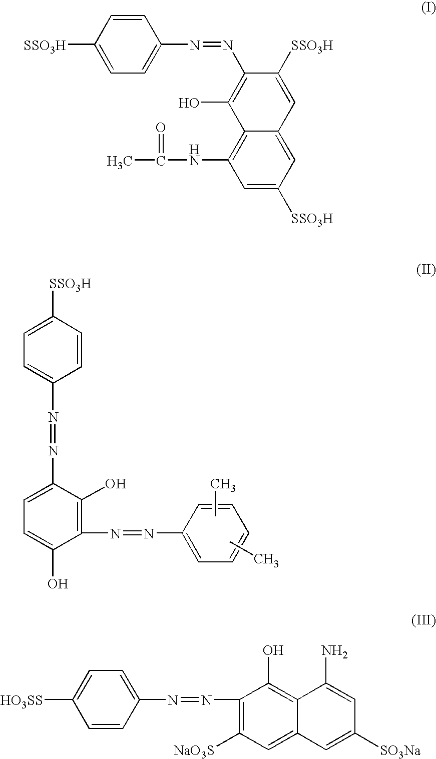

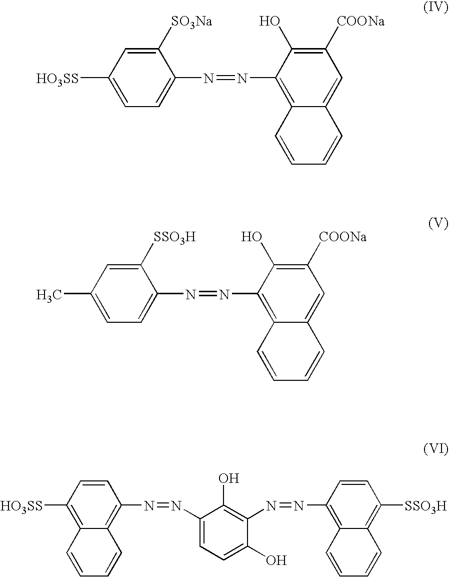

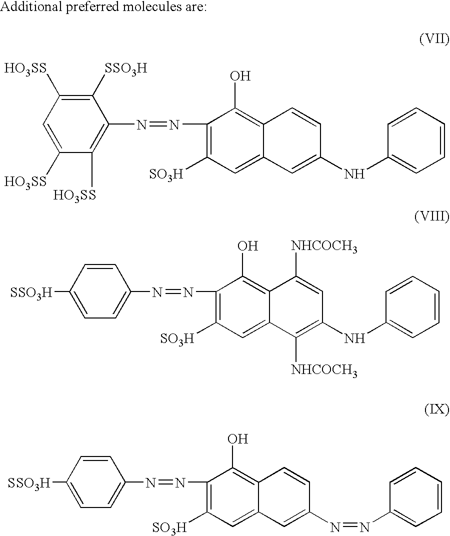

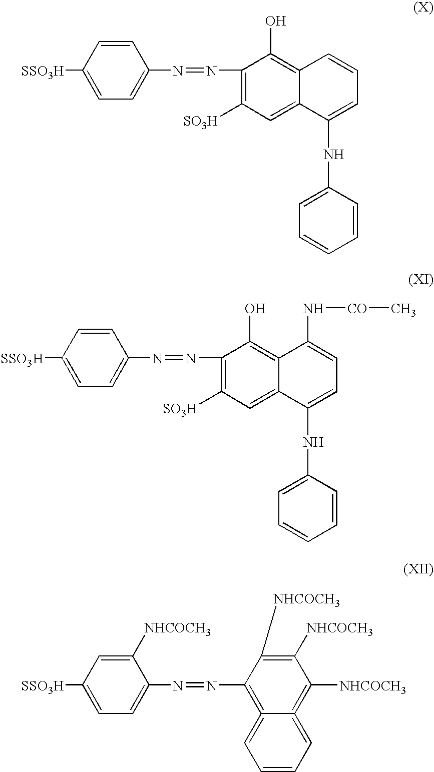

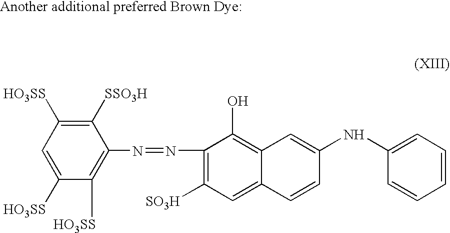

Particularly preferred molecules of the present invention however comprise only one azo group in the molecule.

The non oxidative azo thiosulphato dyes of the present invention can be readily synthesized by diazotization of the S-(4-amino phenyl) thiosulphate by reacting the primary aromatic amine with sodium nitrite in the presence of a mineral acid such as hydrochloric acid to form a diazonium salt, followed by a coupling of the diazonium salt under alkaline conditions (at a pH from about 8 to about 10) with a salt of an aryl amino hydroxy naphthalene sulphonic acid such as phenyl J-acid or phenyl Gamma-acid. Phenyl J acid is a term in the dye art referring to a compound having a molecular formula C₁₆H₁₃NO₄S such as 2-phenylamino-5-naphthol-7-sulphonic acid or 6-phenylamino-1-naphthol-3-sulphonic acid. Phenyl Gamma-acid is term of the dye art referring to a compound having a molecular formula such as C₁₆H₁₃NO₄S such as 2-phenylaminonapothalene-8-hydroxy-6-sulphonic acid.

According to the present invention, each dye is typically utilized at concentrations of from about 0.0001% to about 20% by weight. The exact amount is dependant upon the end shade required. Typically blonde shades comprise from 0.0001% to 1.00%, red shades comprise 0.0001% to 4.00%, brown shades comprise 0.0001% to 4.00% and black shades comprise 0.0001% to 8.00% by weight of the total composition on the hair.

It has also been surprisingly found that the vibrancy of the colour delivered to the hair is dependant upon the molar extinction coefficient of the particular azo thiosulphate dye molecule. It has now been surprisingly found that molecules exhibiting a molar extinction coefficient value of greater than or equal to 20000 mol⁻¹dm³cm⁻¹, preferably at least 30000 mol⁻¹dm³cm⁻¹, more preferably at least 50000 mol⁻¹dm³cm⁻¹ deliver desirable vibrant colour benefits.

It has been further identified that the performance of these cuticle-based non oxidative azo thiosulphate dyes is unexpectedly enhanced when used in combination with an oxidative hair colouring agent, preferably a system of at least one oxidative mainly cortex-based developer and one oxidative mainly cortex-based coupler dye technology. Whilst not being limited by theory it is believed that the cortex-based oxidative dye system delivers an acceptable level of consumer-required grey hair coverage, and an even background colour or “muddiness” to the hair shaft which reduces the sensitivity of the eye to any tonal differences from root to the tip upon application of dye to the hair. The cuticle-based dye(s) of the present invention provide the consumer-required intensity, vibrancy and nuance of color. Moreover the combination of the cuticle-based dye with the mainly cortex-based oxidative dye system delivers the multifaceted, multi-tonal tone, whilst simultaneously ensuring an even colouring coverage along the whole hair shaft (root to tip) and in particular coverage of grey hair.

Moreover the multifaceted, multi-tonal benefits are believed to be due to the presence of oxidative dyes mainly in the cortex at relatively low concentrations and the non oxidative azo thiosulphate dyes of the present invention in the cuticle in relatively high concentrations which result in the consumer eye perceiving varying tonal effects under varying light conditions and delivering a consumer preferred more natural look to the colour of the hair.

Whilst not being bound by theory it is believed that in high intensity light, a high proportion of the incident light is absorbed by both cuticle-based non oxidative azo thiosulphate dye and the mainly cortex-based oxidative dye, but that the majority of reflectance/emittance colour seen by the consumer is due to the high concentrations of bunte azo dye and thus a very vibrant effect and tone is observed.

In lower intensity light conditions, the light is again absorbed by both the mainly cuticle-based azo bunte dyes and the cortex-based oxidative dye, but in this situation, the reflected/emitted light is a more equal combination of the cuticular and cortex emitted/reflected light and hence the color perceived by the observer will be different verses as seen in high intensity light conditions.

Whilst not being bound by theory it is believed that as light strikes the hair surface, the ‘free’, non-matrix-bound hair will be allowed to emit/reflect the high intensity of very vibrant colors from the cuticle with minimal interference and reabsorption of emitted/reflected color by the overall hair matrix. The net effect is more consumer-observed “vibrancy” and intensity. However, for hairs bound in the main body of hair, when they absorb incident light and then reflect/emit the dye color, the surrounding hairs then reabsorb a proportion of this reflected/emitted light. The net effect is less consumer-observed “vibrancy” and intensity, and a different color/shade/tone.

Traditional oxidative hair dyes are prone to “off-toning” as a result of washfade. These hair dyes are made up of multiples of oxidative developers and couplers in pot, pre-application to head and upon oxidative coupling (due to the action of hydrogen peroxide oxidising agent), and application to the hair, the result is a mixture of coupled dimers, trimers and tetramers (wherein two, three and four ringed conjugated dye species are referred to as dimmers, trimers tetramers or possible oligomers or polymers) of varying color tones and shades, sizes, conformations, and water solubility. Post-dyeing, and during the consumer hair washing and drying processes, significant concentrations of varying dimers, trimers and tetramers will elute from the hair at differing rates, dependent on their specific sizes, conformations and water solubility. The net result may be e.g. smaller, more water soluble molecules and hence specific color tones and shades will elute from the hair at a faster rates than the larger, less soluble molecules and hence color tones and shades, and the overall hair color will fade dramatically from its original consumer-target color tone and shade. The final, different color tone and shade will now be more representative of the residual larger, less soluble coloured molecules in the hair. This is a consumer-negative phenomenon known as “off-toning”.

The cuticle-based non oxidative azo thiosulphate dye species together with a system of a one cortex-based oxidative dye developer and one cortex-based oxidative coupler dye precursor in hair offer the significant benefit of the initial and overall colour tone and shade of the newly dyed hair, fading “on-tone” during the consumer hair washing and drying cycle process. The mainly cortex-based one developer and one coupler oxidative coupled dye technologies will deliver significantly reduced numbers of dimers, trimers and tetramers upon coupling, preferably either dimers only, trimers only or tetramers only. Moreover, the one developer and one coupler oxidative coupled dyes can be specifically chosen for their wash-fade durability performance and increased water-insoluble properties, to deliver colour durability over a typical consumer hair washing and drying cycling time period before recolouring is required. Consequently during consumer hair washing, the elution of oxidative dye species from hair can be reduced to a minimum of very select colour tones and shades. In addition, in combination with the durable cuticle-based non oxidative azo thiosulphate dyes, the overall “off-toning” negative is further reduced, with any consumer observed fade being consumer-preferred “on-tone”.

The initial colour of hair just post-dyeing and after a typical consumer wash and dry cycling of approximately 3 to 8 consumer weeks, can be mimicked using the 12 wash and drying cycle described hereinafter which can then be assessed using a HunterLab Lab Scan XE Spectrometer, employing ‘Universe Software’ (herein after referred to as the Hunter). Evaluations are made on the color L,a,b co-ordinates/values i.e. lightness, redness and blueness, and the corresponding L,C,H co-ordinates/values i.e. lightness, chroma and hue. It has been surprisingly found that hair colouring compositions comprising the non oxidative azo thiosulphate molecule having a water solubility of less than 1% at pH 5.5 provide improved durability such that that the change or delta in L, C and H values after a 12 wash and dry cycles (as described hereinafter in the test methods) should not be more than 20, 10, 10 respectively preferably less than 10, 5 and 5 respectively and most preferably less than 6, 0.5 and 0.5 respectively.

Oxidative Hair Colouring Agents

Any oxidative hair coloring agent can be used in the compositions herein. Typically, but without intending to be limited thereby, oxidative hair coloring agents consist essentially of at least two components, which are collectively referred to as dye forming intermediates (or precursors). Dye forming intermediates are non-coloured and can react in the presence of a suitable oxidant to form a conjugated colored molecule.

The dye forming intermediates used in oxidative hair colorants include: aromatic diamines, aminophenols, various heterocycles, (e.g. nitrogen heterocycles) phenols, naphthols and their various derivatives. These dye forming intermediates can be broadly classified as; primary intermediates and secondary intermediates. Primary intermediates, which are also known as oxidative dye developer precursors, are chemical compounds, which become activated upon oxidation by the oxidising agent i.e. hydrogen peroxide and can then react with each other and/or with secondary intermediates or couplers to form coloured conjugated dye complexes. The secondary intermediates, also known as colour modifiers or couplers, are generally colourless molecules, which can form colours in the presence of activated precursors/primary intermediates, and are used with other intermediates to generate specific colour effects or to stabilise the colour.

Primary intermediates or developers, suitable for use in the compositions and processes herein include: aromatic diamines, polyhydric phenols, amino phenols and derivatives of these aromatic compounds (e.g., N-substituted derivatives of the amines, and ethers of the phenols). Such primary intermediates are generally colourless molecules prior to oxidation.

In general terms, oxidative dye primary intermediates or developers are colourless and include those monomeric materials which, on oxidation, form oligomers or polymers having extended conjugated systems of electrons in their molecular structure. Because of the new electronic structure, the resultant oligomers and polymers exhibit a shift in their electronic spectra to the visible range (i.e. 400 nm to 700 nm) and appear coloured. For example, oxidative primary intermediates capable of forming coloured polymers include materials such as aniline, which has a single functional group and which, on oxidation, forms a series of conjugated imines and quinoid dimers, trimers, etc. ranging in colour from green to black. Compounds such as p-phenylenediamine, which has two functional groups, are capable of oxidative polymerization to yield higher molecular weight coloured materials having extended conjugated electron systems.

The primary intermediates can be used herein alone or in combination with other primary intermediates, and one or more can be used in combination with one or more couplers. The choice of primary intermediates and couplers will be determined by the colour, shade and intensity of colouration, which is desired. The primary intermediates and couplers can be used herein, singley or in combination, to provide dyes having a variety of shades ranging from ash blonde to black. Preferred molecules are: resorcinol, p-phenylenediamine, p-aminophenol, 1-naphthol, m-aminophenol, 4-amino-2-hydroxytoluene, N,N, bis(2-hydroxyethyl)-p-phenylenediamine, 2-methyl resorcinol,, phenyl methyl pyrazolone, p-methylaminophenol sulphate, toluene 2,5-diamine sulphate, 2-amino-3-hydroxypyridine, m-phenylenediaminie sulphate, o-aminophenol, dichloro-p-phenylenediamine, 2-amino-4-hydroxyethylaminophenol, 1-hydroxyethyl-4,5-diamino pyrazole, 3-methyl-4-amniophenol and 2-methylnaphthol. These can be used in the molecular form or in the form of peroxide-compatible salts.

The concentration of each oxidative hair colouring agent in the colouring compositions according to the present invention is preferably from about 0.0001% to about 10.00% by weight. The exact amount is dependent upon the end colour and shade required. For example, typically oxidative, permanent blonde shades comprise from 0.0001% to 1.00%, red shades comprise 0.0001% to 4.00%, brown shades comprise 0.0001% to 8.00% and black shades comprise 0.0001% to 4.00% by weight of the total composition on the hair.

For example, low intensity colours such as natural blond to light brown hair shades generally comprise from about 0.0001% to about 5.00%, preferably from about 0.10% to about 2.00%, more preferably from about 0.20% to about 1.00% by weight of colouring composition of total oxidative dyeing agents and may be achieved by the combination of primary intermediates such as 1,4-diamino-benzene, 2,5-diamino toluene, 2,5-diamino-anisole, 4-aminophenol, 2,5-diamino-benzyl alcohol and 2-(2′,5′-diamino)phenyl-ethanol with couplers such as resorcinol, 2-methyl resorcinol or 4-chloro resorcinol.

Similarly, combination of the above primary intermediates with couplers, such as 5-amino-2-methyl phenol and 1,3-diamino-benzene derivatives, such as 2,4-diamino-anisole, at levels of from about 0.50% to about 1.00% of total dyeing agents can lead to medium intensity red colours. High intensity colours such as blue to blue-violet hair shades can be produced by the combination of the above primary intermediates with couplers such as 1,3-diamino-benzene or its derivatives such as 2,5-diamino-toluene at levels of from about 1.00% to about 10.00% by weight of composition of total dyeing agents. Black hair colours can be obtained by combining the aforementioned primary intermediates with couplers such as 1,3-diaminobenzene or its derivatives.

A particularly preferred developer and coupler system for use herein with the azo bunte salts of the present invention are the combinations of p-phenylenediamine with 2-amino-3-hydroxypyridine and the combination of 3-methyl-4-aminophenol with 2-methylnaphthol.

Typically the ratio of the amount by weight of the mono azo thiosulphato dyes of the present invention to the total amount of oxidative dye in the formulation by weight is from 30:0.001 to 30:0.01, preferably from 20:0.01 to 20:0.1, most preferably from 10:1, to 8:1.

Non-Oxidative Dyes and Other Hair Colouring Agents

The hair colouring compositions of the present invention may, in addition to the dyes discussed herein above optionally include other non-oxidative and other dye materials such demi-permanent, semi-permanent, temporary and other dyes. Non-oxidative dyes as defined herein include the so-called ‘direct action dyes’, metallic dyes, metal chelate dyes, fibre reactive dyes, acid dyes, basic dyes, non-ionic dyes, anionic dyes, cationic dyes, HC dyes and other synthetic and natural dyes.

Oxidizing Agents

The hair colouring compositions of the present invention herein preferably comprise at least one oxidizing agent, which may be an inorganic or organic oxidizing agent. The oxidizing agent is preferably present in the colouring composition at a level of from about 0.0001% to about 20%, preferably from about 0.01% to about 10% more preferably from about 1.00% to about 8% by weight of the composition. The oxidizing agents are typically present in formulations comprising oxidative developer and couplers and or to provide lightening of to the hair being coloured.

A preferred oxidizing agent for use herein is an inorganic peroxygen oxidizing agent, which is safe and effective for use and preferably is be soluble in the compositions according to the present invention when in liquid form or in the form intended to be used. Water soluble oxidizing agents as defined herein means agents which have a solubility to the extent of about 10 g in 1000 ml of deionised water at 25° C. (“Chemistry” C. E. Mortimer. 5th Edn. p277).

The inorganic peroxygen oxidizing agents useful herein are generally inorganic peroxygen materials capable of yielding peroxide in an aqueous solution which are well known in the art and include hydrogen peroxide, inorganic alkali metal peroxides such as sodium periodate, sodium perbromate and sodium peroxide, and inorganic perhydrate salt oxidising compounds, such as the alkali metal salts of perborates, percarbonates, perphosphates, persilicates, persulphates and mixtures thereof. These inorganic perhydrate salts may be incorporated as monohydrates, tetrahydrates etc. Highly preferred for use in the compositions according to the present invention is hydrogen peroxide.

In preferred colouring compositions herein the inorganic peroxygen oxidizing agent is present at a level of from about 0.0001% to less than about 6.00%, preferably from about 0.01% to about 4.00%, more preferably from about 1.00% to about 4.00%, more preferably from about 2.00% to about 3.00% by weight of the total composition on hair.

The colouring compositions used in the methods of the present invention can be formulated over a wide pH range, e.g. from about 2 to about 13, but the compositions are typically formulated at high pH, preferably in a pH range of from about 8 to about 12, more preferably from about 9 to about 11, most preferably from about 9.5 to 10.5.

Cosmetically Acceptable Vehicle

The compositions of the present invention typically comprise a cosmetically acceptable vehicle for the non oxidative azo thiosulphate dye molecule in an amount sufficient to carry an effective amount of at least one non oxidative azo thiosulphate molecule onto or into the hair. Typically such amount will range from 0.1% to 99.9% by weight of the composition, preferably from 25% to 99.9%, more preferably from 50% to 99.9%, most preferably from 75% to 99.9%. It has been surprisingly identified that the performance of these dyes is improved when used in Combination with a cosmetically acceptable vehicle and moreover is further enhanced when used in combination with oxidative hair colouring agents.

The vehicle can comprise a semi-solid or liquid cosmetically acceptable vehicle. As used herein, “cosmetically acceptable” means that ingredients which the term describes are suitable for use in contact with the skin or hair of humans without undue toxicity, incompatibility, instability, irritation, allergic response and the like.

The cosmetically acceptable vehicle can itself be inert or it can possess cosmetic benefits of its own. When the compositions are to be applied topically, such cosmetically acceptable vehicles will act as diluents, dispersants, or solvents for the non oxidative azo thiosulphate dyes and any oxidative hair colouring agents that are present, which therefore ensure that they can be applied to and distributed evenly over the hair at an appropriate concentration.

The cosmetically acceptable vehicle will preferably be one which can aid application onto the hair and preferably penetration of the non oxidative azo thiosulphate molecule into the hair. Preferably the cosmetically acceptable vehicle will provide the composition with the desired viscosity at the time of application to a consumer hair to facilitate this process in a quick and clean fashion. Cosmetically acceptable vehicles suitable for use herein alone or in combination include: solvents; thickeners; propellants; fillers; plasticizers; lubricants; surfactants; conditioners and emollients and humectants.

The cosmetically acceptable vehicle of the compositions of the present invention can comprise alone or in combination with other cosmetically acceptable vehicle ingredients, solvents. Solvents suitable for use herein include, but are not limited to: C₁ to C₂₀ mono- or poly-hydric alcohols and their ethers, preferred are C₂ to C₃ mono- and di-hydric alcohols, particularly ethanol, isopropanol, n-propanol, and butanol; propylene glycol; ethylene glycol monoethyl ether; glycerine; methylene chloride; diethylene glycol monobutyl ether; diethylene glycol monoethyl ether; dimethyl sulphoxide; dimethyl formamide; tetrahydrofuran; propylene glycol; and mixtures thereof.

The cosmetically acceptable vehicle of the compositions of the present invention can comprise alone or in combination with other cosmetically acceptable vehicle ingredients, thickening agents. Typically, such thickening agents when present, will be present at a level of from about 0.05% to about 20%, by weight of the composition, preferably from about 0.1% to about 10%, more preferably from about 0.5% to about 5%. It should be understood that under certain circumstances the thickening function maybe accomplished by a material also serving as a silicone or emollient

Thickening agents suitable for use in the compositions herein include, but are not limited to: oleic acid; cetyl alcohol; oleyl alcohol; sodium chloride; cetearyl alcohol; stearyl alcohol; synthetic thickeners such as those available under the tradenames ACULYN (RTM) and SALCARE (RTM) and ELFACOS (RTM), and those cross-linked polyacrylate materials available under the trademark Carbopol (RTM) from the B. F. Goodrich Company; and mixtures thereof. Additional thickening agents suitable for use herein include: sodium alginate; gum arabic; cellulose derivatives; acrylic polymers; polyvinyl-pyrrolidone; gums; clays, and mixtures thereof.

The cosmetically acceptable vehicle of the compositions of the present invention can comprise alone or in combination with other cosmetically acceptable vehicle ingredients, emollients and humectants. Some emollients and humectants which are useful as being all or part of the vehicle herein include, but are not limited to: esters; fatty alcohols and acids; polyols; hydrocarbons; non-volatile silicones; waxes; animal fats; vegetable oils; and mixtures thereof.

The cosmetically acceptable vehicle of the composition according to the present invention may comprise alone or in combination with other vehicle ingredients at least one hair-conditioning agent. The conditioning agent is preferably present at a level of from about 0.0001% to about 25.00%, preferably from about 1.00% to about 20.00%, more preferably from about 5.00% to about 20.00% and especially from about 5.00% to about 15.00%, by weight of the composition.

Suitable conditioning agents for use herein include, but are not limited to, cationic surfactants, cationic polymers, soluble and insoluble silicones, non-volatile hydrocarbons, saturated C14-C22 straight chain fatty alcohols, non-volatile hydrocarbon esters, and mixtures thereof. Other suitable conditioning agents are disclosed in WO95/20939 and WO96/32919, which are incorporated herein by reference.

Preferred conditioning agents for use herein include cationic surfactants, cationic polymers, soluble and insoluble silicone conditioning agents and saturated C14-C22 straight chain fatty alcohols and mixtures thereof. Especially preferred for use herein is a mixture of cationic polymer, non-volatile silicone and C14-C22 straight chain fatty alcohols.

The cosmetically acceptable vehicle of the composition of the present invention may comprises alone or in combination with a other cosmetically acceptable vehicle ingredients a surfactant system. Suitable surfactants generally have a lipophilic chain length of from about 8 to about 22 carbon atoms and can be selected from anionic, cationic, nonionic, amphoteric, zwitterionic surfactants and mixtures thereof. Particularly preferred are cationic surfactants, nonionic surfactants and mixtures thereof. Suitable surfactants for use herein are disclosed in WO98/27945, which is incorporated herein by reference in its entirety.

Optional Materials

The compositions of the present invention typically further comprise a number of other components commonly utilized in hair care compositions such as shampoos, conditioners, styling aids and colourants which are well known to those skilled in the art. These optional materials can be added to the compositions herein described each at a level of from about 0.0001% to about 5%, preferably from about 0.01% to about 3%, more preferably from about 0.05% to about 2% by weight of composition.

Such materials include water-soluble or solubilizable preservatives such as EDTA, Euxyl (RTM) K400, natural preservatives such as benzyl alcohol, benzoic acid, sodium benzoate and 2-phenoxyethanol; antioxidants such as sodium sulphite, hydroquinone, sodium bisulphite, sodium metabisulphite and thyoglycolic acid, sodium dithionite, erythrobic acid and other mercaptans; dye removers such as oxalic acid, sulphated castor oil, salicylic acid and sodium thiosulphate; H₂ O₂ stabilisers; anti-bacterial agents; low temperature phase modifiers such as ammonium ion sources (e.g. NH₄ Cl); ammonia; metal ion sequestrants, chelants such as polycarboxylates, polyphosphonates, and their amino derivatives, EDTA, ethylenediaminie disuccinic acid and water softening agents such as sodium citrate.

Method of Use

The composition of the present invention may be provided as a single composition containing all the necessary colouring ingredients. However typically when the colouring composition comprises oxidative colouring agents and oxidizing agents, it is preferably provided in the form of two components, one of which contains the oxidative colouring agent and the second of which contains the oxidizing agent. The non oxidative azo thiosulphate dyes of the present invention may be present in either of these components or as a separate third component. When the composition is provided in the form of two components these may be made up into the composition before application to the hair or applied separately to form a single composition on the hair. Percentages and amounts when discussed in this specification refer to percentages and amounts in the final composition on the hair.

As a consequence the colouring composition can be provided as a single pack or in kit form as separately packaged components to maintain stability.

In order to facilitate the easy application of the hair composition of the present invention onto the hair of the user it is preferable that the applied composition has a certain viscosity. This can be achieved by supplying each component such that they have the desired viscosity prior to mixing and maintaining this viscosity after mixing. These formulations are typically provided as a cream and are referred to as a thick, thick, thick system. Alternatively, the two components may be provided as relatively thin fluids which contain materials which on mixing cause the viscosity of the resultant mixture to rapidly increase to the desired level. These systems are referred to as a thin, thin, thick systems and are typically gel type compositions. The present invention finds application as either of these composition types.

A particular advantage of the non oxidative azo thiosulphate dyes of the present invention is the absence of the requirement of a pre-reduction process of the hair, prior to the application of the dyes. This thus avoids unnecessary packaging and steps for the consumer to follow and reduces the damage to the hair.

Test Methods

LAB/LCH Value Determination

Apparatus: Hunter Lab Scan XE Spectrophotometer employing the Universe software.

1. Click on the ‘Universe’ icon to initialize the software

2. Select the appropriate size of port and ensure it is securely fitted to the magnetic holder on the top of the equipment

3. Calibrate the sensor click on the ‘standardize’ icon. Ensure that the software settings for the ‘area view’ and ‘port size’ correspond to the hardware settings. Area view should always be≦port size.

For the LabScan XE

| Area View (mm) | Port Size (mm) |
|---|---|
| 3.2 | 5.0 |
| 6.4 | 10.0 |
| 12.7 | 17.0 |
| 25.4 | 30.5 |
| 44.5 | 50.8 |

The standard settings should be as follows:

| Area View (mm) | Port Size (mm) |
|---|---|
| 12.7 | 17.0 |

4. Adjust the dial on the right side of the LabScan to fix the diameter of the beam. The beam diameter should always be slightly bigger than the diameter of the port: choose the stop position, which corresponds to the beam diameter one size larger than the port size.

5. Follow the on screen instructions to calibrate the instrument, using the tiles from the accessory box belonging to that piece of equipment.

After the sensor has been successfully standardized readings can commence.

Re-standardization should be carried out each time the port size is changed, ensuring the appropriate ‘port size’ and ‘area view’ settings are used.

6. Select the ‘Read’ icon to set measurement method. E.g. average method, Continuous readings, Automatic saving etc.

7. Place the hair switch to be measured in an appropriately sized switch holder and make sure the springs pull the hair taut and flat against the holder. The hair should be mounted on the white side of the switch holder, to ensure measurement against a white background. Place the sample face down over the measuring port and select ‘Read’. (A total of eight readings are taken for normal tests; 4 on one side of the switch, and four on the other side, moving the hair switch along its length between readings).

8. The display of results can be altered by selecting ‘Active View’ and adjusting the appropriate fields. This active view can be printed by selecting the Print option.

Calculation of Molar Extinction Coefficient: (Molar Asorbtivity)

The calculations are based on the Beer Lambert Law:

A=□bc

Where A is absorbance (no units, since A=log₁₀P₀/P). Where P₀, is the radiant power beam of monochromatic radiation directed at a sample solution, and P is the radiant power of the beam of leaving the sample.

□ is the molar absorbtivity with units of L mol⁻¹ cm⁻¹

b is the path length of the sample—that is, the path length of the cuvette in which the sample is contained, expressed in centimetres.

c is the concentration of the compound in solution, expressed in mol L⁻¹

A=□bc tells us that absorbance depends on the total quantity of the absorbing compound in the light path through the sample.

By a simple algebraic rearrangement we are ample to calculate the molar absorbtivity, or as it is referred to in this patent, the Molar Extinction Coefficient.

□=A/bc

Experimental data obtained for this patent ensured that the concentration of the dye (in De-Ionised Water) was such that the maximum absorbance was as near to, but not greater, than 1.0, as the Beer Lambert Law is not obeyed at high concentrations.

Water Solubility Method:

1. Prepare a “batch” solution of dye in De-Ionised (DI) water; ensuring the dye material is completely dissolved within the water (Visual inspection). E.g.: 0.090 gL⁻¹, stir solution at room temperature for 60 minutes.

2. Use “batch” solution to prepare a series of samples of varying dye concentration by diluting samples of the batch solution.

E.g.

Sample 1=1 ml batch in 50 ml DI water

Sample 2=2 ml batch in 50 ml DI water

Sample 3=5 ml batch in 50 ml DI water

Run UV-Vis spectra, noting UV-Vis machine used (Machine used for Patent data: Zeiss: Specord UV-Vis S10, and Aspect Plus Software Package). Run spectra scanning in the range between 300-1000 nm. Ensure that the concentration of the dye (in De-Ionised Water) used is such that the maximum absorbance is not greater, than 1.0, as the Beer Lambert Law is not obeyed at high concentrations.

Ensure AT LEAST 3 spectra are obtained that have clear spectral definition (i.e. Clearly define the maximum absorption peak), whilst ensuring the maximum absorbance is less than 1.0.

3. Select a absorbance wavelength near, but not at the maximum absorbance peak, e.g. Maximum absorption at 420 nm, select 400 nm, and record the absorbance at this selected wavelength for the 3+ sample you ran at the varying concentrations.

4. Plot a Concentration Curve using above data (X-Axis: Concentration gL⁻¹, Y-Axis: Absorbance at selected wavelength). Obtain the regression line equation, i.e.: y=mx+c, where m represents the gradient of the line and c represents the intercept on the y axis.

5. Prepare a saturated solution of dye in DI water. Typically load a large excess of dye into a small volume of water, e.g. 5 g in 20 g of DI water. Stir at room temperature for upto 2 hrs. Inspect saturated solution solution. If precipitate observed assume solution is saturated. If no precipitate observed reload further dye, e.g. additional 5 g, and repeat 2 hr stir and further observations. Continue additional loading and stirring of dye until precipitate observed.

6. Take sample of saturated solution and filter through fine filter paper, e.g. filter via in-line syringe filter of 0.45 micro meters. Use this filtrate to prepare a solution of saturated solution in DI water that gives a maximum absorbance peak below 1.0 E.g. 0.1 ml saturated solution in 1 L of DI water.

7. Run UV-Vis spectra, noting UV-Vis machine used (Machine used for Patent data: Zeiss: Specord UV-Vis S10, and Aspect Plus Software Package). Run spectra scanning in the range between 300-1000 nm. Record the absorption of the saturated solution at the selected wavelength.

8. Taking this absorption figure as the y-axis value, substitute into y=mx+c equation obtained at step 4, to obtain the x-axis value, i.e the concentration of the saturated solution.

9. Using the x-axis saturated solution concentration value obtained at step 8, ensure this value is scaled correct according to the dilution of saturated solution to water concentration used at step 6.

10 This value obtained is the water solubility of the saturated solution, which is taken to be the maximum water solubility of the dye, expressed in gL⁻¹.

Wash Fade Test Method

Hair Switches:

The hair switches may be bleached-damaged Yak (pigmentless) or human hair, or permed-damaged Yak or human hair, where human hair may be 100% grey or pigmented. Pre-damaged, pigmentless switches show the greatest amount of dye loss from washing, while pigmented switches show the likely effect of the underlying natural pigment in human hair (natural hair melanin) on the final colour fade result.

8 Switches minimum should be used for each prototype. When switches are prebleached-damaged or pre-permed-damaged, they should be treated according to the manufacturers' instruction. The current recommended bleaching product is L'Oreal Excellence 01 and recommended perming product is Zotos Perming solution.

Switch Dyeing:

1. Mix dyes, hydrogen peroxide and perfume as appropriate, ensure complete mixing.

2. Apply 2 g of colourant per 1 gram of hair.—If dyeing a large number of switches simultaneously, e.g. five 6 g switches, apply half the of the dye mixture initially (30 g) and half once the first mixture has been massaged into the hair to prevent it dripping off. Ensure the syringe you use is the correct size to provide an accurate result.

3. Apply colourant with a syringe using a zigzag action down the length of the hair.

4. Massage WELL to ensure even coverage, check that centre of switch is completely saturated.

5. Wrap switches in clingfilm and place in 30° C. oven for 30 min.

6. Rinse hair for 1 min. Rinse using “shower method”:-

Hold shower head at an angle at top of switch/switches.

Allow water to flow down through switch and out at the end (like a ‘funnel’). Do not touch the switch until squeezing excess water from it.

Switch Washing:

1. Thoroughly wet switches for 30 sec.

2. Apply 0.1 g shampoo (Pantene Classic Care) per gram of hair (e.g. 0.15 ml shampoo for each 1.5 g switch). Use a 2 ml syringe—larger syringes will increase inaccuracy.

3. Apply shampoo with a syringe using a zigzag action down the length of the hair.

4. Milk shampoo into hair for 30 secs, ensuring even application.

Milk at a speed of less than 30 strokes/60 seconds (i.e. 1 stroke every 2 seconds).

Milk using a ‘2 fingers and thumb’ action.

5. Rinse hair for 1 min using “shower method”:-

Hold showerhead at an angle at top of switch/switches.

Allow water to flow down through switch and out at the end (like a ‘funnel’). Do not touch the switch until squeezing excess water from it.

6. Apply 0.1 g shampoo per gram of hair (e.g. 0.15 ml shampoo for each 1.5 g switch)

7. Apply shampoo with a syringe using a zigzag action down the length of the hair.

8. Milk shampoo into hair for 30 secs, ensuring even application.

Milk at a speed of less than 30 strokes/60 seconds (i.e. 1 stroke every 2 seconds) using a ‘2 fingers and thumb’ action.

9. Rinse hair for 1 min., using a “shower method”:

Hold shower head at an angle at top of switch/switches.

Allow water to flow down through switch and out at the end (like a ‘funnel’). Do not touch the switch until squeezing excess water from it.

Assessments:

Take L, a, b reading for all dry switches initially (before any washing)

Retain 2 switches

For the remaining switches repeat washing for 3 cycles

Take L, a, b reading for all dry switches

For the remaining switches repeat washing for 3 cycles

Take L, a, b reading for all dry switches

For the remaining switches repeat washing and conditioning after washing for 6 cycles

Take L, a, b reading for all dry switches

The L.a.b data recorded can be converted into L. C and H values via simple mathematical calculations. In general these are carried out using computer spreadsheets (e.g. Microsoft Excel). The aforementioned calculations are:

$$\text{Choma:} C = \sqrt{{a}^{2} ⊕ {b}^{2}}$$

$$\text{Hue:} {h}^{0} = {tan}^{- 1} ( \frac{b}{a} )$$

The delta L, C and H values are then determined after the wash protocol has been completed. Initial Uptake dL, dC and dH is the difference between the starting substrate and the final dyed switch. Wash Fade d/dL, d/dC and d/dH is the difference between the final washed switch (after 12 wash and dry cycles) and the originally dyed switch.

The delta L, C and H calculations are:

$$Delta L : d L = {L}_{Trial} - {L}_{Reference}$$

$$Delta C : dC = {C}_{Trial} - {C}_{Reference}$$

$$Delta H : dH = \sqrt{{dE}^{2} - d {L}^{2} - {dC}^{2}}$$

$$Where dE = \sqrt{d {L}^{2} + {da}^{2} + {db}^{2}}$$

$$in which da = {a}_{Trial} - {a}_{Reference}$$

$$and db = {b}_{Trial} - {b}_{Reference}$$

After each cycle the hair switches are dried using following drying protocol.

Drying Protocol with Hot Air Drying

1. Lay the hair on clean paper towel and blot the hair gently.

2. Blow dry with a blow dryer on high heat/high speed for a total of 3 minutes,

1 min with finger separation, (No Brushing)

Rotate the switches

1 min with finger separation, (No Brushing)

Using a vented brush blow dry for 1 minute until dry, making sure not to brush faster than 20-30 reps/min. Do not force the brush through the hair. The hairdryer (Babyliss Lightweight Professional model 1015 (1400 W)) should be placed within 12 cm (˜5″) of the switches.

Damaging Hair via Perming Test Method

1. Hang switches over sink using ergonomically approved switch holders. Maximum 6×1.5 g switches, 6×4 g switches or 5×6 g switches.

2. Apply perm solution to switches and thoroughly saturate; use 2 g of perm solution per 1 g of hair, e.g. Apply 3 g of perming solution to 1.5 g hair switch.

3. Lay switches in plastic tray, wrap loosely in cling film and leave for 15 minutes in a Fume Hood.

4. Hang switches over sink. Turn on water to 37° C.+/−2° C. Remember to always turn on the cold tap first to prevent scalding. Flow Rate=6/7L/min. Record type of water used, e.g. Tap water.

5. Rinse hair switches thoroughly for 2 min (1 min each side), lightly massaging hair to ensure water penetration.

6. Squeeze/towel (tissue towel) dry switches.

7. Apply neutralising solution to switches and thoroughly saturate; use 2 g of neutraliser solution per 1 g of hair, e.g. Apply 3 g of neutraliser solution to 1.5 g hair switch.

8. Lay switches in plastic tray, wrap loosely in cling film and leave for 5 minutes in a Fume Hood.

9. Hang switches over sink. Turn on water to 37° C.+/−2° C. Remember to always turn on the cold tap first to prevent scalding. Flow Rate=6/7L/min. Record type of water used, e.g. Tap water.

10. Rinse hair switches thoroughly for 2 min (1 min each side) lightly massaging hair to ensure water penetration.

11. Apply 0.1 ml Pantene Classic Care Shampoo per 1 g hair, e.g. 0.15 ml per 1.5 g hair switch.

Apply shampoo with a syringe using a zigzag action down the length of the hair.

12. Milk shampoo into hair for 30 seconds, ensuring even application.

13. Rinse hair switches thoroughly for 2 min (1 min each side) lightly massaging hair to ensure water penetration.

14. Squeeze/towel (tissue towel) dry switches.

15. Place Hair Switches into large Pyrex beaker and fill with De-Ionised (DI) water. Ensure that the switches are not over-packed into the beaker and that the beaker is clearly labelled with relevant details.

16. Place beaker of hair switches into safety approved and calibrated oven at 50° C. for minimum of 12 hours, this is to ensure the majority of the perming solution has been removed from the hair.

17. Remove beaker with care and discard water to drain.

18. Rinse hair switches thoroughly for 2 min (1 min each side) lightly massaging hair to ensure water penetration.

19. Dry Switches using Hair Dryer. (Record wattage and heat/speed setting)

### EXAMPLES

Mono azo thiosulphato dye (1)

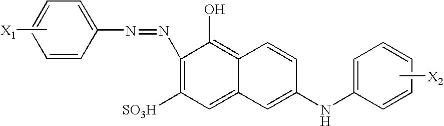

Wherein X₁ is SSO₃H and X₂ is H.

The below data is all generated on 100% Grey, Perm Damaged Hair.

Starting Substrate Data: L.C.H values

| | Substrate | Substrate | Substrate |
|---|---|---|---|
| Hair Substrate | L | Chroma | Hue |
| 100% Grey: Permed | 75.32 | 28.89 | 80.79 |

| | | Wash | | Wash | | Wash |
|---|---|---|---|---|---|---|
| | Uptake | Fade | Uptake | Fade | Uptake | Fade |
| Molecule(s) | dL | d/dL | dC | d/dC | dH | d/dH |
| Formula 1 | −32.80 | 5.77 | 56.89 | 0.08 | 48.00. | 0.4 |
| Formula 1 & | −54.98 | 2.90 | 17.52 | 3.2 | 23.96 | 1.64 |
| PPD & AHP | | | | | | |
| References | | | | | | |
| L'Oreal Feria: Ruby | −56.47 | 7.18 | −7.72 | 1.1 | 20.89 | 2.68 |
| Fusion | | | | | | |
| Schwarzkopf LIVE: | −56.25 | 9.11 | −10.63 | 3.83 | 18.23 | 0.4 |
| Red Night | | | | | | |
| Goldwell Elumen | −41.11 | 1.71 | 30.89 | 0.3 | 35.77 | 0.23 |
| RR @ ALL (Red Dye) | | | | | | |

Wherein:
PPD = p-phenylamine-diamine
AHP = 2-amino-3-hydroxypyridine
and are used at a ratio of 2:1 and wherein the ratio of formula 1 to PPD and AHP is 8.89:1.

Below is an example of a red hair dye formulation 1:

I Dye base:

| q.s.50 | Water |
|---|---|
| 0.1 | Tetra sodium EDTA |
| 0.2 | L-ascorbic acid |
| 2.5 | Dipropyleneglycol |
| 1.9552 | 50% Acetic acid |
| 0.3 | p-Phenylenediamine |
| 0.15 | 2-Amino-3-Hydroxypyridine |
| 24.75 | Emulsion base |
| 0.5 | Decyl glucoside |
| 4.0 | Bunte Salt Red |
| 5.5 | Ammonium hydroxide |

II Emulsion Base

Weight % in use

| q.s | water |
|---|---|
| 1.5 | ceteareth 25 |
| 2.25 | cetyl alcohol |
| 2.25 | stearyl alcohol |
| 0.06 | sodium benzoate |
| 0.07 | phenoxyethanol |
| 0.08 | benzyl alcohol |
| 0.02 | tetrasodium EDTA |
| 2.0 | silicone (DC Q2-8220 from Dow Corning) |

III Hydrogen Peroxide Emulsion Base

Weight % in use

| q.s | water |
|---|---|
| 4.2 | ceteareth 25 |
| 6.25 | cetyl alcohol |
| 6.25 | stearyl alcohol |

IV Hydrogen Peroxide Cream

Weight %

| 36 | Hydrogen peroxide emulsion base |
|---|---|
| 17.7 | 35% hydrogen peroxide |
| q.s | water |

The dye emulsion base is prepared by a one pot process as follows:-

One-Pot Process for Making Dye Cream Emulsion

1. Add water to vessel. Heat to above the melt temperature of the fatty alcohols with agitation.

2. Add Fatty Alcohols and any Ethoxylated Fatty Alcohols, e.g. Ceteareth-25, Cetyl, Stearyl and Steareth-2, and allow to melt. Increase agitation.

3. Continue mixing with shear.

4. Begin cooling with shear adding preservatives at appropriate temperature.

5. During cooling add silicone with mixing until homogeneous.

6. Cool to room temperature.

The hydrogen peroxide cream is also prepared similarly using a one pot process. All 3 components are thoroughly mixed before application to hair.

The above formulation provided excellent red colour, grey coverage and root to tip coverage, which was maintained on tone during the post dyeing wash cycle.

### Examples of Formulation: Thickened Aqueous Solution

| | 2 | 3 | 4 | 5 |
|---|---|---|---|---|
| Sodium sulphite | 0.1 | 0.1 | 0.1 | 0.1 |
| Ascorbic Acid | 0.1 | 0.1 | 0.1 | 0.1 |
| Citric Acid | 1.0 | 1.0 | 1.0 | 1.0 |
| Ammonia (30% active) | 6.0 | 6.0 | 6.0 | 6.0 |
| Acrylates Copolymer | 2.4 | 2.4 | 2.4 | 2.4 |
| (Aculyn ® 33A) | | | | |
| Oleth 5 | 1.0 | 1.0 | 1.0 | 1.0 |
| Oleth 2 | 0.8 | 0.8 | 0.8 | 0.8 |
| Oleic Acid | 0.9 | 0.9 | 0.9 | 0.9 |
| Cocamidopropylbetaine | 3.0 | 3.0 | 3.0 | 3.0 |
| DTPMP tetrasodium salt) | 2.5 | — | 2.5 | — |
| DEPTA(pentasodium salt) | — | — | 0.5 | 1.0 |
| EDDS (trisodium salt) | — | 1.0 | — | 1.0 |
| Para-phenylene Diamine | 0.8 | 0.5 | 0.6 | 0.5 |
| 2-Amino-3-hydroxypyridine | 0.2 | 0.3 | 0.2 | 0.1 |
| Formula 1 | 4.0 | 4.0 | 4.0 | 4.0 |
| Hydrogen Peroxide 35% | 8.6 | 8.6 | 8.6 | 13 |
| active) | | | | |
| Behentrimonium Chloride | 0.5 | 0.5 | 1.5 | 2.0 |
| Dicetyldimonium Chloride | 0.2 | 0.2 | 0.7 | 0.2 |
| Acrylates Steareth-20 Methacrylate Copolymer | 0.5 | 0.5 | — | 1.0 |
| (Aculyn ® 22) | | | | |
| PropyleneGlycol | 8.2 | 8.2 | 8.2 | 8.2 |
| Ethoxy Diglycol | 4.2 | 4.2 | 4.2 | 4.2 |
| Adjust to pH 10 | qs | qs | qs | qs |
| Water | qs | qs | qs | qs |

What is claimed is:

1. A durable hair colouring composition comprising at least one non oxidative azo thiosulphate dye molecule, wherein said molecule has a water solubility of less than 1% at pH 5.5 and said durable composition has a delta L of less than 20, a delta C of less than 10 and a delta H of less than 10 after a 12 wash and dry cycle wherein said composition further comprises a cosmetically acceptable vehicle.

2. A hair colouring composition according to claim 1, wherein said dye has the following formula and the salts thereof: 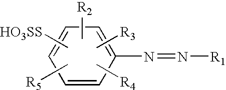
wherein thiosulphato benzene ring is substituted by R2, R3, R4 and R5, wherein each of R2, R3, R4, and R5 are independently selected from:
1) hydrogen,
2) C1-C10 linear or branched alkyl, C1-C10 linear or branched substituted alkyl,
3) C1-C10 linear or branched alkenyl,
4) Aryl,
5) C1-C8 non aryl ring systems or C1-C8 substituted non aryl rings,
6) auxochromes,
7) nitro, nitroso, thiosulphato, sulphonate, alkyl sulphone and alkenyl sulphone or sulphato alkyl sulphone and
8) mixtures thereof.
wherein R1 is selected from:
1) hydrogen,
2) C1-C10 linear or branched alkyl, C1-C10 linear or branched substituted alkyl,
3) C1-C10 linear or branched alkenyl,
4) Aryl,
5) C1-C8 non aryl ring systems or substituted C1-C8 non aryl rings,
6) auxochromes,
7) and mixtures thereof.

3. A hair colouring composition according to claim 2, wherein R1 is: 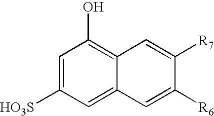
wherein R6 and R7 are independently selected from:
1) hydrogen,
2) C1-C10 linear or branched alkyl, C1-C10 linear or branched substituted alkyl,
3) C1-C10 linear or branched alkenyl,
4) Aryl,
5) C1-C8 non aryl ring systems or C1-C8 substituted non aryl rings,
6) Auxochromes,
7) and mixtures thereof.

4. A hair colouring composition according to claim 2, wherein R2, R3, R4 and R5 are hydrogen and R6 or R7 is a phenylamine or a substituted phenylamine.

5. A hair colouring composition according to claim 1, further comprising at least one oxidative dye coupler and at least one oxidative dye developer.

6. A hair colouring composition according to claim 1, wherein said composition further comprises at least one oxidative dye coupler and at least one oxidative dye developer, wherein the amount by weight of said non oxidative azo thiosulphato dye to the total amount of oxidative dye in the formulation by weight is from about 30:00.001 to about 30:0.1.

7. A hair colouring composition according to claim 1, wherein said composition further comprises a chelant.

8. A hair coluring composition according to claim 6 wherein wherein said composition further comprises at least one oxidative dye coupler and at least one oxidative dye developer, wherein the amount by weight of said non oxidative azo thiosulphato dye to the total amount of oxidative dye in the formulation by weight is from about 30:00.001 to about 30:0.1.

9. A durable and vibrant hair colouring composition comprising at least one non oxidative azo thiosulphate dye molecule, wherein said molecule has a molar extinction coefficient of greater than 20000 mol⁻¹dm³cm⁻¹, wherein said composition further comprises a cosmetically acceptable vehicle.

10. A hair colouring composition according to claim 9 wherein said molecule has a molar extinction coefficient of greater than 30000 mol⁻¹dm³cm⁻¹

11. A hair colouring composition according to claim 9 wherein said molecule has a molar extinction coefficient of greater than 50000 mol⁻¹dm³cm⁻¹

12. A hair colouring composition according to claim 9, wherein said dye has the following formula and the salts thereof: 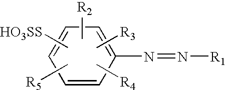
wherein thiosulphato benzene ring is substituted by R2, R3, R4 and R5, wherein each of R2, R3, R4, and R5 are independently selected from:
1) hydrogen,
2) C1-C10 linear or branched alkyl, C1-C10 linear or branched substituted alkyl,
3) C1-C10 linear or branched alkenyl,
4) Aryl,
5) C1-C8 non aryl ring systems or C1-C8 substituted non aryl rings,
6) auxochromes,
7) nitro, nitroso, thiosulphato, sulphonate, alkyl sulphone and alkenyl sulphone or sulphato alkyl sulphone and
8) mixtures thereof.
wherein R1 is selected from:
1) hydrogen,
2) C1-C10 linear or branched alkyl, C1-C10 linear or branched substituted alkyl,
3) C1-C10 linear or branched alkenyl,
4) Aryl,
5) C1-C8 non aryl ring systems or substituted C1-C8 non aryl rings,
6) auxochromes,
7) and mixtures thereof.

13. A hair colouring composition according to claim 9, wherein R1 is: 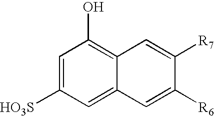
wherein R6 and R7 are independently selected from:
1) hydrogen,
2) C1-C10 linear or branched alkyl, C1-C10 linear or branched substituted alkyl,
3) C1-C10 linear or branched alkenyl,
4) Aryl,
5) C1-C8 non aryl ring systems or C1-C8 substituted non aryl rings,
6) auxochromes,
7) and mixtures thereof.

14. A hair colouring composition according to claim 9, wherein R2, R3, R4 and R5 are hydrogen and R6 or R7 is a phenylamine or a substituted phenylamine.

15. A hair colouring composition according to claim 9, further comprising at least one oxidative dye coupler and at least one oxidative dye developer.

16. A hair colouring composition according to claim 9, wherein said composition further comprises at least one oxidative dye coupler and at least one oxidative dye developer, wherein the amount by weight of said non oxidative azo thiosulphato dye to the total amount of oxidative dye in the formulation by weight is from about 30:00.001 to about 30:0.1.

17. A hair colouring composition according to claim 9, wherein said composition further comprises a chelant.

18. A method of colouring hair comprising the steps of: applying a hair colouring composition according to claim 2 to hair for a period of from about 1 minute to about 1 hour and subsequently rinsing said composition from the hair.
